(19) United States
(12) Patent Application Publication
(10) Pub. No.: US 2025/0127798 A1
(43) Pub. Date: Apr. 24, 2025

(54) METHODS FOR THE TREATMENT OF TINNITUS

(71) Applicant: BUZZELET DEVELOPMENT AND TECHNOLOGIES LTD., Or Akiva (IL)

(72) Inventors: Noa RAZ, Gizo (IL); Aharon EYAL, Jerusalem (IL)

(21) Appl. No.: 18/836,385

(22) PCT Filed: Feb. 6, 2023
(86) PCT No.: PCT/IB2023/051045

§ 371 Date: Aug. 7, 2024

Related U.S. Application Data
(60) Provisional application No. 63/307,654, filed on Feb. 8, 2022.

(51) Int. Cl. A61K 31/00; A61K 31/01; A61K 31/015; A61K 31/045; A61K 31/352; A61P 27/16

(52) U.S. Cl. CPC A61K 31/658; A61K 31/01; A61K 31/015; A61K 31/045; A61K 31/352; A61P 27/16

(57) ABSTRACT

Provided are compositions and methods for use in the treatment of tinnitus or a symptom thereof in a subject in need thereof, comprising administering to the subject a therapeutically effective amount of a composition comprising cannabidiol (CBD), cannabidiolic acid (CBDA) or a combination thereof; and a mixture of at least three selected terpenes.

### FIELD OF THE INVENTION

The present invention relates to the field of medical treatment, and more specifically to methods for the treatment of tinnitus and symptoms thereof using cannabidiol (CBD) and/or cannabidiolic acid (CBDA) and a mixture of at least three selected terpenes.

### BACKGROUND OF THE INVENTION

Tinnitus is a condition characterized by phantom noises (such as ringing, buzzing, hissing or the like), in one or both ears, which is not caused by an external sound. It is especially common in older adults.

Tinnitus may be caused by an underlying condition, such as age-related hearing loss, an ear injury or infection, a head or neck trauma, or a problem with the circulatory system. Certain medications, such as some nonsteroidal anti-inflammatory drugs, antibiotics, diuretics, antidepressants, antimalarial drugs and others may cause or worsen tinnitus.

Although the severity of tinnitus may be reduced in some cases by treatment of any underlying conditions, there is currently no safe and effective treatment for tinnitus itself.

### SUMMARY OF THE INVENTION

According to an aspect of some embodiments of the present invention, there is provided a method for treating tinnitus or a symptom thereof in a subject in need thereof, the method comprising administering to the subject a composition comprising a therapeutically effective amount of:
(i) cannabidiol (CBD), cannabidiolic acid (CBDA) or a combination thereof; and
(ii) a mixture of terpenes selected from the group consisting of:
   (a) at least three selected from the group consisting of pinene, myrcene, limonene, linalool, caryophyllene, humulene, nerolidol and bisabolol;
   (b) at least three selected from the group consisting of pinene, myrcene, caryophyllene, nerolidol and bisabolol;
   (c) at least three selected from the group consisting of pinene, ocimene, linalool, caryophyllene, humulene and nerolidol;
   (d) at least three selected from the group consisting of pinene, myrcene, linalool, caryophyllene, humulene and nerolidol;
   (e) at least three selected from the group consisting of pinene, myrcene, terpineol, borneol, caryophyllene and nerolidol;
   (f) at least three selected from the group consisting of myrcene, ocimene, linalool, terpineol, caryophyllene, nerolidol and bisabolol;
   (g) at least three selected from the group consisting of ocimene, linalool, terpineol, nerolidol and caryophyllene;
   (h) at least three selected from the group consisting of pinene, limonene, terpinolene, caryophyllene and bisabolol;
   (i) at least three selected from the group consisting of pinene, limonene, terpinolene, geraniol, caryophyllene and bisabolol;
   (j) at least three selected from the group consisting of pinene, sabinene, limonene, terpinolene, eucalyptol, borneol and caryophyllene;
   (k) at least three selected from the group consisting of pinene, sabinene, limonene, eucalyptol, caryophyllene and bisabolol;
   (l) at least three selected from the group consisting of pinene, sabinene, limonene, eucalyptol, borneol and caryophyllene;
   (m) at least three selected from the group consisting of pinene, terpinolene, eucalyptol, linalool, caryophyllene and bisabolol; and
   (n) at least three selected from the group consisting of pinene, carene, eucalyptol, linalool, geraniol, limonene and caryophyllene.

### DETAILED DESCRIPTION OF THE INVENTION

The present invention relates to methods for the treatment of tinnitus and symptoms thereof using cannabidiol (CBD) and/or cannabidiolic acid (CBDA) and a mixture of at least three selected terpenes

Unless otherwise defined, all technical and scientific terms used herein have the same meaning as commonly understood by one of ordinary skill in the art to which this invention belongs.

The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

As used herein, the term “treating” includes ameliorating, mitigating, and reducing the instances of a disease or condition, or the symptoms of a disease or condition.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

As used herein, the term “therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

Unless otherwise indicated, all numbers expressing quantities, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the terms “water content” and “moisture content” are used interchangeably.

Unless indicated otherwise, percent is weight percent and ratio is weight/weight ratio.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a method for treating tinnitus or a symptom thereof in a subject in need thereof, the method comprising administering to the subject a composition comprising a therapeutically effective amount of:
(i) cannabidiol (CBD), cannabidiolic acid (CBDA) or a combination thereof; and
(ii) a mixture of terpenes selected from the group consisting of:
   (a) at least three selected from the group consisting of pinene, myrcene, limonene, linalool, caryophyllene, humulene, nerolidol and bisabolol;
   (b) at least three selected from the group consisting of pinene, myrcene, caryophyllene, nerolidol and bisabolol;
   (c) at least three selected from the group consisting of pinene, ocimene, linalool, caryophyllene, humulene and nerolidol;
   (d) at least three selected from the group consisting of pinene, myrcene, linalool, caryophyllene, humulene and nerolidol;
   (e) at least three selected from the group consisting of pinene, myrcene, terpineol, borneol, caryophyllene and nerolidol;
   (f) at least three selected from the group consisting of myrcene, ocimene, linalool, terpineol, caryophyllene, nerolidol and bisabolol;
   (g) at least three selected from the group consisting of ocimene, linalool, terpineol, nerolidol and caryophyllene;
   (h) at least three selected from the group consisting of pinene, limonene, terpinolene, caryophyllene and bisabolol;
   (i) at least three selected from the group consisting of pinene, limonene, terpinolene, geraniol, caryophyllene and bisabolol;
   (j) at least three selected from the group consisting of pinene, sabinene, limonene, terpinolene, eucalyptol, borneol and caryophyllene;
   (k) at least three selected from the group consisting of pinene, sabinene, limonene, eucalyptol, caryophyllene and bisabolol;
   (l) at least three selected from the group consisting of pinene, sabinene, limonene, eucalyptol, borneol and caryophyllene;
   (m) at least three selected from the group consisting of pinene, terpinolene, eucalyptol, linalool, caryophyllene and bisabolol; and
   (n) at least three selected from the group consisting of pinene, carene, eucalyptol, linalool, geraniol, limonene and caryophyllene.

According to some embodiments, the mixture of terpenes comprises at least three, at least four, at least five, of the terpenes from a specified group.

According to some embodiments, each terpene in the mixture of terpenes comprises about 0.05-1.5 wt % of the total composition, about 0.05-0.1 wt % of the total composition, about 0.05-0.5 wt % of the total composition, about 0.1-0.4 wt % of the total composition, or about 0.2-0.3 wt % of the total composition. According to some such embodiments, each terpene in the mixture comprises about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45 or 1.5 wt % of the total composition. According to some embodiments, each of the terpenes is present at the same concentration. According to some embodiments, each terpene may independently be present at any concentration in the range of about 0.05-1.5 wt % of the total composition.

According to some embodiments, a total weight of the mixture of terpenes is about 0.5-5.0 wt % of the total composition, about 0.5-4.0 wt % of the total composition, about 1.0-3.0 wt % of the total composition. According to some such embodiments, a total weight of the mixture of terpenes is about 0.5, 1.0, 1.5, 2.0, 2.5, 3.0, 3.5, 4.0, 4.5 or 5 wt % of the total composition.

According to some embodiments, administering the composition comprising the mixture of terpenes (a) to (g) is carried out at nighttime.

According to some embodiments, administering the composition comprising the mixture of terpenes (h) to (n) is carried out during daytime.

According to some embodiments, the composition further comprises a cannabinoid selected from the group consisting of cannabichromene (CBC), cannabigerol (CBG), cannabinol (CBN), cannabidivarin (CBDV), tetrahydrocannabivarin (THCV), tetrahydrocannabiniolic acid (THCA), tetrahydrocannabiniol (THC) and combinations thereof.

According to some embodiments, administration is by a route selected from the group consisting of sublingual, oral, transdermal, nasal, buccal, inhalation, aural, or ocular administration. According to some such embodiments, the composition is provided for administration in the form of drops, such as ear drops.

According to some embodiments, the composition is administered to the subject four times per day, three times per day, twice a day, once a day, six times per week, five times per week, four times per week, three times per week, twice per week, once weekly, three times per month, twice per month, or once monthly. According to some embodiments, the composition is administered to the subject for a period of at least one week, at least two weeks, at least three weeks, at least one month, at least six weeks or at least two months. According to some embodiments, the composition is administered to the subject at least once a day for at least one week, such as one week, two weeks, three weeks, one month, six weeks or two months. According to some embodiments, the composition is administered to the subject at least twice a day for at least one week, such as one week, two weeks, three weeks, one month, six weeks, or two months.

### EXAMPLES

### Example 1: Compositions Tested

TABLE 1

| | | Delivery | Delivery | Delivery |
|---|---|---|---|---|
| Cannabinoid(s) | Terpenes | form | route | time |
| Cannabidiol | Pinene, myrcene and | Drops | Buccal | Night |
| | caryophyllene | | | |
| Cannabidiol | Limonene, linalool | Drops | Sublingual | Night |
| | and humulene | | | |
| Cannabidiol | Pinene, ocimene, | Aerosol | Nasal | Night |
| | caryophyllene and | | | |
| | nerolidol | | | |
| Cannabidiolic | Myrcene, linalool, | Drops | Ocular | Night |
| acid | humulene and | | | |
| | nerolidol | | | |
| Cannabidiolic | Terpineol, borneol | Drops | Sublingual | Night |
| acid | nerolidol and | | | |
| | myrcene | | | |
| Cannabidiol | Myrcene, ocimene, | Aerosol | Nasal | Night |
| and cannabidiolic | terpineol, neorlidol | | | |
| acid | and bisabolol | | | |
| Cannabidiol | Ocimene, linalool | Drops | Buccal | Night |
| and cannabidiolic | and nerolidol | | | |
| acid | | | | |
| Cannabidiol | Limonene, | Vaporizer | Inhalation | Morning |
| and cannabidiolic | terpinolene, | liquid | | |
| acid | caryophyllene and | | | |
| | bisabolol | | | |
| Cannabidiol | Geraniol, | Vaporizer | Inhalation | Morning |
| | caryophyllene and | liquid | | |
| | bisabolol | | | |
| Cannabidiol | Sabine, limonene | Vaporizer | Inhalation | Morning |
| | and eucalyptol | liquid | | |
| Cannabidiol | Sabine, caryophyllene | Tablet | Oral | Morning |
| | and bisabolol | | | |
| Cannabidiol | Pinene, sabinene, | Tablet | Oral | Morning |
| | eucalyptol, borneol | | | |
| | and caryophyllene | | | |
| Cannabidiol | Pinene, terpinolene, | Tablet | Oral | Morning |
| | eucalyptol, linalool, | | | |
| | caryophyllene and | | | |
| | bisabolol | | | |
| Cannabidiol | Carene, eucalyptol, | Tablet | Oral | Morning |
| | geraniol and limonene | | | |
| Cannabidiol | Pinene, borneol and | Drops | Aural | Morning + |
| and cannabidiolic | caryophyllene | | | Night |
| acid | | | | |
| Cannabidiol and | Pinene, linonene, | Drops | Sublingual | Morning |
| cannabidiolic | eucalyptol, borneol | | | |
| acid | and caryophyllene | | | |

Male and female subjects in the age group 18-80 suffering from tinnitus are selected and divided equally into a test group and a control group. Each test subject is matched to a control subject.

A composition comprising a carrier and active agents as defined in Table 1 is administered to each of the test subjects according to the delivery route and schedule specified in the table.

Placebo compositions comprising carrier alone are administered to each of the control subjects according to the delivery route and schedule used for the matched test subject.

An average decrease in the symptoms associated with tinnitus at the end of the treatment period as reported by the test subjects is significantly greater than that reported by control subjects.

1. A method for treating tinnitus or a symptom thereof in a subject in need thereof, the method comprising administering to the subject a therapeutically effective amount of a composition comprising:
(i) cannabidiol (CBD), cannabidiolic acid (CBDA) or a combination thereof; and
(ii) a mixture of terpenes selected from the group consisting of:
(a) at least three selected from the group consisting of pinene, myrcene, limonene, linalool, caryophyllene, humulene, nerolidol and bisabolol;
(b) at least three selected from the group consisting of pinene, myrcene, caryophyllene, nerolidol and bisabolol;
(c) at least three selected from the group consisting of pinene, ocimene, linalool, caryophyllene, humulene and nerolidol;
(d) at least three selected from the group consisting of pinene, myrcene, linalool, caryophyllene, humulene and nerolidol;
(e) at least three selected from the group consisting of pinene, myrcene, terpineol, borneol, caryophyllene and nerolidol;
(f) at least three selected from the group consisting of myrcene, ocimene, linalool, terpineol, caryophyllene, nerolidol and bisabolol;
(g) at least three selected from the group consisting of ocimene, linalool, terpineol, nerolidol and caryophyllene;
(h) at least three selected from the group consisting of pinene, limonene, terpinolene, caryophyllene and bisabolol;
(i) at least three selected from the group consisting of pinene, limonene, terpinolene, geraniol, caryophyllene and bisabolol;
(j) at least three selected from the group consisting of pinene, sabinene, limonene, terpinolene, eucalyptol, borneol and caryophyllene;
(k) at least three selected from the group consisting of pinene, sabinene, limonene, eucalyptol, caryophyllene and bisabolol;
(l) at least three selected from the group consisting of pinene, sabinene, limonene, eucalyptol, borneol and caryophyllene;
(m) at least three selected from the group consisting of pinene, terpinolene, eucalyptol, linalool, caryophyllene and bisabolol; and
(n) at least three selected from the group consisting of pinene, carene, eucalyptol, linalool, geraniol, limonene and caryophyllene.

2. The method of claim 1, wherein each terpene in said mixture of terpenes comprises 0.05-0.5 wt % of the total composition.

3. The method of claim 1, wherein a total weight of said mixture of terpenes is 0.5-5 wt % of the total composition.

4. The method of claim 1, wherein administering the composition comprising the mixture of terpenes (a) to (g) is carried out at nighttime.

5. The method of claim 1, wherein administering the composition comprising the mixture of terpenes (h) to (n) is carried out during daytime.

6. The method of claim 1, wherein said composition further comprises a cannabinoid selected from the group consisting of cannabichromene (CBC), cannabigerol (CBG), cannabinol (CBN), cannabidivarin (CBDV), tetrahydrocannabivarin (THCV), tetrahydrocannabiniolic acid (THCA), tetrahydrocannabiniol (THC) and combinations thereof.

7. The method of claim 1, wherein said administration is by a route selected from the group consisting of sublingual, oral, transdermal, nasal, buccal, inhalation, aural, or ocular administration.

8. The method of claim 7, wherein said composition is provided for administration in the form of drops.

9. The method of claim 1, wherein said administration is repeated at least once a day for at least a week.

10. The method of claim 9, wherein said administration is repeated at least twice a day for at least a week.
